(19) United States
(12) Patent Application Publication
(10) Pub. No.: US 2023/0194404 A1
(43) Pub. Date: Jun. 22, 2023

(54) DETERMINING FLUID PROPERTIES

(71) Applicant: Saudi Arabian Oil Company, Dhahran (SA)

(72) Inventors: Thomas Hillman, Missouri City, TX (US); Miguel Angel Gonzalez Cadena, Houston, TX (US)

(21) Appl. No.: 17/553,229

(22) Filed: Dec. 16, 2021

(51) Int. Cl. G01N 11/04; G01N 33/28

(52) U.S. Cl. CPC G01N 11/04; G01N 33/2823

(57) ABSTRACT

A system includes a pump to circulate a flow of a fluid from a fluid source through a flow conduit assembly at unique flow rates; a flow meter fluidly coupled within the flow conduit assembly; a coiled tubing assembly fluidly coupled within the flow conduit assembly and including one or more tubing coils; a pressure sensor coupled to the flow conduit assembly and configured to measure a fluid pressure drop across the coiled tubing assembly; and a control system communicably coupled to the flow meter and the pressure sensor. The control system is configured to identify each of the unique flow rates of the flow of fluid through the coiled tubing assembly; identify, for each unique flow rate, the fluid pressure drop across the coiled tubing assembly; for each unique flow rate and corresponding measured fluid pressure drop, determine flow rate-dependent properties of the fluid; and generate a rheogram from the determined flow rate-dependent properties.

### TECHNICAL FIELD

The present disclosure describes apparatus, systems, and methods for determining fluid properties and, more particularly, determining properties of a drilling fluid.

### BACKGROUND

During drilling of a wellbore, drilling fluid (or “drilling mud”) is used to lubricate a drilling bit and carry cuttings up the annulus between the drill string and the wall. It is important for the drilling fluid properties to be maintained in order to avoid problems with torque, stuck pipe, cutting carrying capacity, fluid losses, and wellbore stability and control. During the drilling process, properties of the fluid may be affected by many factors such as shearing, variation in temperature, variation in pressure, and chemical interactions. The fluid properties should be maintained in order to perform efficiently, therefore additives may need to be added to the fluid to maintain fluid properties. A key parameter of the drilling fluid is its viscosity, which affects its ability to transport cuttings away from the bit. However, the viscosity in these fluids is shear rate dependent, therefore a complete rheogram of the fluid needs to be determined. This information is used as input to hydraulic modeling calculations. These measurements can be done by sampling the fluid from the available mud pits, and the measurements are typically carried out in a mobile laboratory. There are disadvantages to this process because the measurements are not made in real time, it is done by a human technician and relies on the skill and experience of the technician carrying out the measurements.

### SUMMARY

In an example implementation, a system for determining one or more properties of a fluid includes a pump configured to circulate a flow of a fluid from a fluid source through a flow conduit assembly at a plurality of unique flow rates, the flow conduit assembly including an inlet configured to fluidly coupled to the fluid source at a first location and an outlet configured to fluidly coupled to the fluid source at a second location; a flow meter fluidly coupled within the flow conduit assembly; a coiled tubing assembly fluidly coupled within the flow conduit assembly between the inlet and the outlet, the coiled tubing assembly including one or more tubing coils; a pressure sensor coupled to the flow conduit assembly and configured to measure a fluid pressure drop across the coiled tubing assembly; and a control system communicably coupled to the flow meter and the pressure sensor. The control system is configured to perform operations including identifying each of the plurality of unique flow rates of the flow of fluid through the coiled tubing assembly measured by the flow meter; identifying, for each unique flow rate, the fluid pressure drop across the coiled tubing assembly measured by the pressure sensor; for each unique flow rate and corresponding measured fluid pressure drop, determining one or more flow rate-dependent properties of the fluid; and generating a rheogram from the determined one or more flow rate-dependent properties.

In an aspect combinable with the example implementation, the fluid includes a drilling fluid.

In another aspect combinable with any of the previous aspects, the first location is at or near a bottom of a drilling fluid tank, and the second location is at or near a top of the drilling fluid tank.

In another aspect combinable with any of the previous aspects, each of the plurality of unique flow rates includes a plurality of unique steady state flow rate.

In another aspect combinable with any of the previous aspects, the flow meter includes a Coriolis meter and the pressure sensor includes a differential pressure sensor.

In another aspect combinable with any of the previous aspects, the operation of determining one or more flow rate-dependent properties of the fluid includes determining, for each unique flow rate and corresponding measured fluid pressure drop, a shear rate of the fluid; and determining, for each unique flow rate and corresponding measured fluid pressured drop, a shear stress of the fluid.

In another aspect combinable with any of the previous aspects, the operation of determining the shear stress of the fluid includes calculating the shear stress based at least in part on an inner diameter of the one or more tubing coils and the measured fluid pressure drop.

In another aspect combinable with any of the previous aspects, the operation of calculating the shear stress includes solving

$${τ}_{w} = \frac{D}{4} \frac{d p}{d l} ,$$

where τ_{w} is the shear stress, D is the inner diameter of the one or more tubing coils, and

$$\frac{d p}{d l}$$

is the measured fluid pressure drop per length of the coiled tubing assembly.

In another aspect combinable with any of the previous aspects, the operation of determining the shear rate of the fluid includes calculating the shear rate based at least in part on the calculated shear stress, the inner diameter of the one or more tubing coils, a velocity of the flow of the fluid, and the measured fluid pressure drop.

In another aspect combinable with any of the previous aspects, the operation of calculating the shear rate includes solving

$${γ}_{w} = \frac{1}{4} \left[3 + \frac{d \left(l n \frac{8 v}{D}\right)}{d \left({τ}_{w}\right)}\right] \left(\frac{8 v}{D}\right) ,$$

where γ_{w} is the shear rate, τ_{w} is the shear stress, D is the inner diameter of the one or more tubing coils, and v is the velocity of the fluid.

In another aspect combinable with any of the previous aspects, the coiled tubing assembly includes a first coiled tubing assembly and the one or more tubing coils includes one or more first tubing coils, the system further including a second coiled tubing assembly fluidly coupled within the flow conduit assembly between the inlet and the outlet, the second coiled tubing assembly including one or more second tubing coils.

In another aspect combinable with any of the previous aspects, the first and second coiled tubing assemblies are fluidly coupled in series.

In another aspect combinable with any of the previous aspects, an inner diameter of the one or more second tubing coils is different than an inner diameter of the one or more first tubing coils.

In another aspect combinable with any of the previous aspects, the control system is configured to perform operations including identifying each of the plurality of unique flow rates of the flow of fluid through the first and second coiled tubing assemblies as measured by the flow meter; identifying, for each unique flow rate, the measured fluid pressure drop across each of the first and second coiled tubing assemblies by the pressure sensor; for each unique flow rate and corresponding measured fluid pressure drop across the first coiled tubing assembly, determining a first set of one or more flow rate-dependent properties of the fluid; for each unique flow rate and corresponding measured fluid pressure drop across the second coiled tubing assembly, determining a second set of one or more flow rate-dependent properties of the fluid; and generating a rheogram from the determined first and second sets of one or more flow rate-dependent properties.

In another example implementation, a method for determining one or more properties of a fluid includes circulating, with a pump, a flow of a fluid from a fluid source through a flow conduit assembly at a plurality of unique flow rates, the flow conduit assembly including an inlet configured to fluidly coupled to the fluid source at a first location and an outlet configured to fluidly coupled to the fluid source at a second location; circulating, with the pump, the flow of fluid at each of the plurality of unique flow rates through one or more tubing coils of a coiled tubing assembly fluidly coupled within the flow conduit assembly between the inlet and the outlet; during circulation of the flow of fluid at each of the plurality of unique flow rates through the one or more tubing coils of the coiled tubing assembly, measuring the unique flow rate with a flow meter coupled within the flow conduit assembly; during circulation of the flow of fluid at each of the plurality of unique flow rates through the one or more tubing coils of the coiled tubing assembly, measuring a fluid pressure drop across the coiled tubing assembly with a pressure sensor coupled to the flow conduit assembly; for each unique flow rate and corresponding measured fluid pressure drop, determining one or more flow rate-dependent properties of the fluid; and generating a rheogram from the determined one or more flow rate-dependent properties.

In an aspect combinable with the example implementation, the fluid includes a drilling fluid.

In another aspect combinable with any of the previous aspects, the first location is at or near a bottom of a drilling fluid tank, and the second location is at or near a top of the drilling fluid tank.

In another aspect combinable with any of the previous aspects, each of the plurality of unique flow rates includes a plurality of unique steady state flow rate.

In another aspect combinable with any of the previous aspects, the flow meter includes a Coriolis meter and the pressure sensor includes a differential pressure sensor.

In another aspect combinable with any of the previous aspects, determining one or more flow rate-dependent properties of the fluid includes determining, for each unique flow rate and corresponding measured fluid pressure drop, a shear rate of the fluid; and determining, for each unique flow rate and corresponding measured fluid pressured drop, a shear stress of the fluid.

In another aspect combinable with any of the previous aspects, determining the shear stress of the fluid includes calculating the shear stress based at least in part on an inner diameter of the one or more tubing coils and the measured fluid pressure drop.

In another aspect combinable with any of the previous aspects, calculating the shear stress includes solving

$${τ}_{w} = \frac{D}{4} \frac{d p}{d l} ,$$

where τ_{w} is the shear stress, D is the inner diameter of the one or more tubing coils, and

$$\frac{d p}{d l}$$

is the measured fluid pressure drop per length of the coiled tubing assembly.

In another aspect combinable with any of the previous aspects, determining the shear rate of the fluid includes calculating the shear rate based at least in part on the calculated shear stress, the inner diameter of the one or more tubing coils, a velocity of the flow of the fluid, and the measured fluid pressure drop.

In another aspect combinable with any of the previous aspects, calculating the shear rate includes solving

$${γ}_{w} = \frac{1}{4} \left[3 + \frac{d \left(l n \frac{8 v}{D}\right)}{d \left({τ}_{w}\right)}\right] \left(\frac{8 v}{D}\right) ,$$

where γ_{w} is the shear rate, τ_{w} is the shear stress, D is the inner diameter of the one or more tubing coils, and v is the velocity of the fluid.

In another aspect combinable with any of the previous aspects, the coiled tubing assembly includes a first coiled tubing assembly and the one or more tubing coils includes one or more first tubing coils, and the method further includes circulating, with the pump, the flow of fluid at each of the plurality of unique flow rates through one or more second tubing coils of a second coiled tubing assembly fluidly coupled within the flow conduit assembly between the inlet and the outlet.

In another aspect combinable with any of the previous aspects, the first and second coiled tubing assemblies are fluidly coupled in series.

In another aspect combinable with any of the previous aspects, an inner diameter of the one or more second tubing coils is different than an inner diameter of the one or more first tubing coils.

Another aspect combinable with any of the previous aspects further includes during circulation of the flow of fluid at each of the plurality of unique flow rates through the one or more first tubing coils of the first coiled tubing assembly and the one or more second tubing coils of the second coiled tubing assembly; measuring the unique flow rate with the flow meter coupled within the flow conduit assembly; during circulation of the flow of fluid at each of the plurality of unique flow rates through the one or more first tubing coils of the first coiled tubing assembly, measuring a first set of fluid pressure drops across the first coiled tubing assembly with the pressure sensor coupled to the flow conduit assembly; during circulation of the flow of fluid at each of the plurality of unique flow rates through the one or more second tubing coils of the second coiled tubing assembly, measuring a second set of fluid pressures drop across the second coiled tubing assembly with the pressure sensor coupled to the flow conduit assembly; for each unique flow rate and corresponding measured first and second sets of fluid pressure drops, determining one or more flow rate-dependent properties of the fluid; and generating a rheogram from the determined one or more flow rate-dependent properties.

Implementations of systems and methods for determining fluid properties according to the present disclosure may include one or more of the following features. For example, a rheometer system according to the present disclosure can provide real time (for example, during a drilling process) fluid property determinations of a drilling fluid at a well site (rather than a laboratory). A rheometer system according to the present disclosure can also provide more accurate pressure drop measurements to determine such fluid properties while still having a compact physical design by using coiled tubing assemblies. As another example, a rheometer system according to the present disclosure can provide for an automated process and system for accurately determined flow rate-dependent fluid properties.

The details of one or more implementations of the subject matter described in this disclosure are set forth in the accompanying drawings and the description below. Other features, aspects, and advantages of the subject matter will become apparent from the description, the drawings, and the claims.

### BRIEF DESCRIPTION OF THE DRAWINGS

FIG. 1 is a schematic diagram of a rheometer system for determining fluid properties of a drilling fluid according to the present disclosure.

FIG. 2 is a flowchart of an example method for determining fluid properties of a drilling fluid according to the present disclosure.

FIG. 3 shows an example rheogram as determined by an example method for determining fluid properties of a drilling fluid according to the present disclosure.

FIG. 4 is a schematic illustration of an example control system of a rheometer system for determining fluid properties of a drilling fluid according to the present disclosure.

### DETAILED DESCRIPTION

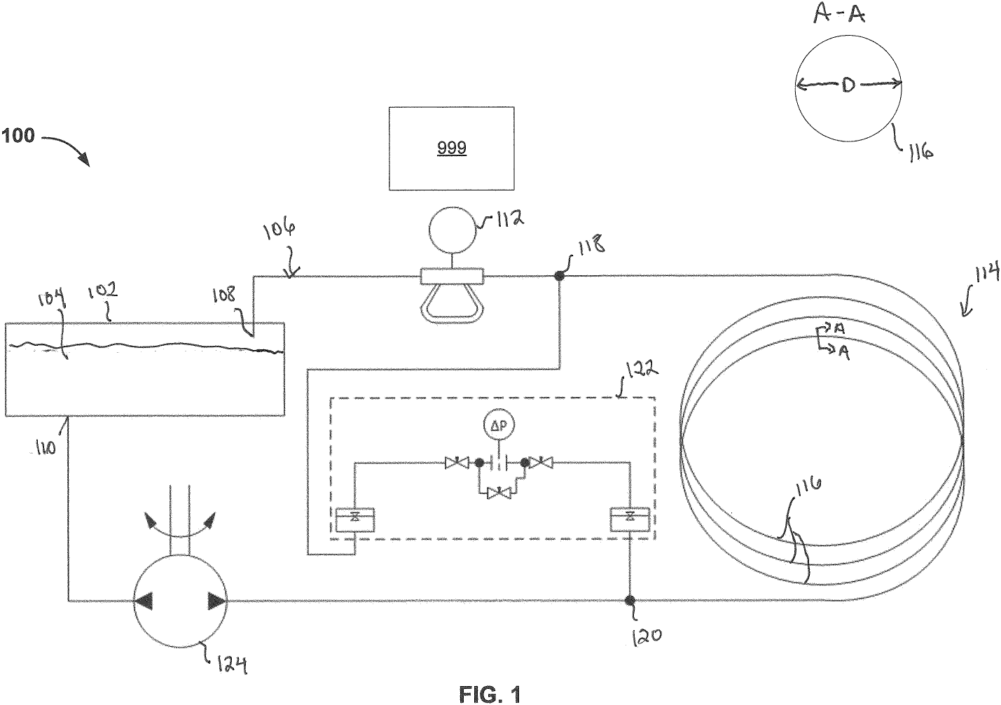

FIG. 1 is a schematic diagram of a rheometer system 100 for determining fluid properties of a drilling fluid according to the present disclosure. Generally, the rheometer system 100 is operable to measure one or more properties of a fluid, such as a drilling fluid, and determine one or more rheological properties (for example, flow rate dependent properties) of the fluid while the fluid is being used, such as in a drilling operation at a well site. The rheometer system 100, in some aspects, can be used in an analysis of drilling mud during a wellbore drilling process. In some aspects, the rheometer system 100 can provide real time (in other words, during the drilling process) drilling fluid rheology analysis at a drilling site with a mobile system that can be moved from well site to well site. For example, rather than relying on samples of used drilling fluid that are brought to a lab (remote from a drilling site) for rheological analysis.

In the example implementation of the rheometer system 100 shown in FIG. 1, a fluid source 102 contains a fluid 104. In example aspects, the fluid source 102 can be a drilling fluid source, or “mud tank” 102 and the fluid 104 is drilling mud 104 (fluid used in the drilling process to lubricate a drill bit and circulate cuttings to the surface out of the wellbore). The rheometer system 100, as shown, can be fluidly connected to the mud tank 102 (at a drilling site) at an inlet 110 of a flow conduit assembly 106 (for example, at a sump or bottom of the mud tank 102) and at an outlet 108 of the flow conduit assembly 106 (for example, at a top of the mud tank 102). The flow conduit assembly 106, in this example, can be comprised of multiple conduit sections (tubing sections) as well as, for instance, valves, couplings, fluid filters, backflow preventers, and other flow control components).

The rheometer system 100 also includes one or more pumps 124 (for example, centrifugal, submersible such as within the mud tank 102, or otherwise) that are operable to circulate a flow of the drilling mud 104 through the flow conduit assembly 106 from the inlet 110 to the outlet 108 (and through the other components of the rheometer system 100). In some aspects, the one or more pumps 124 can be operated at a constant speed to produce (in a steady state condition) a constant flow rate of the drilling mud 104 through the flow conduit assembly 106.

As shown, the flow conduit assembly 106 includes or is coupled with a coiled tubing assembly 114 that includes one or more coils 116 between a fluid input 120 of the coiled tubing assembly 114 and a fluid output 118 of the coiled tubing assembly 114. In this example, the number of coils 116 is three; however, alternative implementations of the rheometer system 100, the coiled tubing assembly 114 can have more or fewer coils 116. In some aspects, by using the coiled tubing assembly 114, problems associated with limitations of scale of a mobile rheometer system can be avoided. For example, conventional rheometer systems rely on measuring pressure drops in a fluid through straight lengths of pipe across a range of flow rates. However, pressure differences between the inlet and outlet of a pipe section can be difficult to measure as the pipe length becomes shorter and as the pipe diameter increases, because the pressure difference becomes very small. Practical limitations on the length of the device in the field, therefore, put a constraint on the pipe length and by consequence also the pipe diameter. Aside from limiting the range of shear rates that can be developed, this size constriction also fixes the measurement error based on the ratio between the measured pressure drop in the pipe and the absolute error of a differential pressure transducer that measures the pressure drop. In other words, the signal to noise ratio from the differential pressure transducer is low.

By using the coils 116 of the coiled tubing assembly 114, a longer flow length of pipe through which the drilling mud 104 can be circulated to avoid such problems, while still maintaining a mobile and compact design of the system 100 (to allow for real time analysis at a well site). For example, the use of the coils 116 allows the rheometer system 100 to be capable of measuring a wider range of shear conditions of the drilling mud 104, with less error from restriction on pipe length. The coils 116 can also allow for the length of the measurement section to be increased substantially as compared to conventional, straight pipe rheometer systems, within a small footprint. Also, increase in the measurement section length (in other words, straight line tubing length of the coiled tubing assembly 114) can correspond to an increase in pressure drop between the fluid output 118 and the fluid input 120, thereby providing for greater measurement accuracy. Further, the coils 116 can also have a larger diameter tubing as compared to convention, straight pipe rheometer systems (while not increasing the footprint), thereby allowing for a larger range of shear rates that can be achieved to improve pressure measurement accuracy.

Further, as the number of coils 116 in the coiled tubing assembly 114, a total length of measurement section of the coiled tubing assembly 114 (between the fluid input 120 and the fluid output 118) also increases. As the measurement section increases, a pressure drop measured across the coiled tubing assembly 114 of the drilling mud 104 as it circulates through the flow conduit assembly 106 also increases. This increase can, in turn, reduce precision requirements of a pressure sensor 122 (for example, a differential pressure transducer) that is positioned in the flow conduit assembly 106 to measure the pressure drop across the coiled tubing assembly 114. However, if the pressure drop becomes too high (with increasing number of coils 116), then it can become difficult to pump the drilling mud 104 at several, unique flow rates (for example, steady state rates). Thus, a number of coils 116 can be optimized based on, for example, available pump power, tube diameter (of the coils 116), and precision of the pressure sensor 122 to determine an appropriate total length of the coiled tubing assembly 114 between the fluid input 120 and the fluid output 118.

In some aspects, the coiled tubing assembly 114 can comprise multiple (two or more) sets of coils 116. In some aspects, each sets of coils 116 can be connected in series to form the coiled tubing assembly 114. In such aspects, there can be also multiple pressure sensors 122, with each pressure sensor 122 fluidly connected across a particular set of coils 116. Thus, in some aspects, several (two or more) different pressure drop measurements can be obtained (one from each set of coils 116) for a single, steady state flow rate of the drilling mud 104 through the coiled tubing assembly 114. In some aspects, such as when each set of coils 116 has a unique inner diameter, the different pressure drops are also unique and correspond to a particular combination of steady state flow rate of the drilling mud 104, inner diameter of the coils 116, and length of the particular coiled section.

As shown in this example, one or more of the coils 116 (and, in some aspects, all of the coils 116), can have a bend radius (later described as R) and an inner diameter, D. As shown in FIG. 1, section A-A is a cross-section of a coil 116, which shows the inner diameter, D.

In this example implementation, a flow meter 112 is positioned in the flow conduit assembly 106 downstream of the coiled tubing assembly 114. In some aspects, the flow meter 112 can be a Coriolis meter that accurately measures a flow rate of the fluid 104 as it is circulated (by the pump 124) through the coiled tubing assembly 114. Alternatively, the flow meter 112 can be a differential pressure flow meter, magnetic flow meter, multiphase flow meter, turbine flow meter, ultrasonic flow meter, or vortex flow meter. After the flowing fluid 104 passes through the flow meter 112, it can be deposited back into the fluid source 102 through outlet 108.

As shown in this example, a control system (or controller) 999 can be part of the rheometer system 100 or in communication with one or more components of the rheometer system 100, such as the pressure sensor 122, flow meter 112, and/or the pump 124 (for example, to control start and stop or speed of the pump 124). In some aspects, the control system 999 comprises a micro-processor based controller that includes one or more hardware processors, one or more memory modules, and a communication interface (to communicate data and/or commands between it and the components of the rheometer system 100). Alternatively, the control system 999 can be a mechanical control system, electro-mechanical control system, or even a pneumatic control system. In any event, control system 999 is operable to receive data from one or more components of the rheometer system 100 and determine one or more flow rate-dependent properties of the fluid 104 from such data.

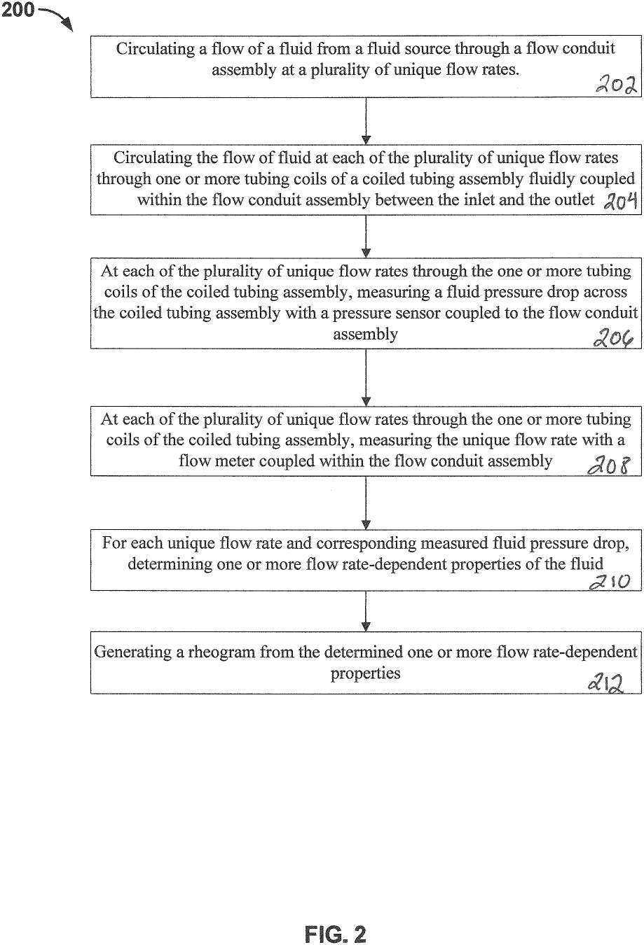

FIG. 2 is a flowchart of an example method 200 for determining fluid properties of a drilling fluid according to the present disclosure. In some aspects, for example, method 200 can be performed by or with the rheometer system 100 as shown in FIG. 1, or another rheometer system according to the present disclosure. In some aspects, for example, the rheometer system 100 can be installed at a wellbore drilling site, where the inlet 110 is plumbed to a mud tank 102, and the outlet 108 is also plumbed to the mud tank 102 (or other tank). The mud tank 102 can enclose a volume of drilling mud 104 (for example, being returned from the drilling process or saved from an earlier drilling process). In some aspects, one or more steps of method 200 can be performed by or with the control system 999.

Method 200 can begin at step 202, which includes circulating a flow of a fluid from a fluid source through a flow conduit assembly at a plurality of unique flow rates.. For example, pump 124 can be operated to circulate the drilling mud 104 from the mud tank 102, through the inlet 110, and into the flow conduit assembly 106. In some aspects, the pump 124 can be operated (for example, turned on, speed adjusted, etc.) by the control system 999. In some aspects, the pump 124 flows the drilling mud 104 through the flow conduit assembly 106 at a particular (for example, pre-determined or programmed) flow rate for a particular time period, such as to at least achieve a steady state flow at the particular rate while measurements in steps 206 and 208 occur. The pump 124 then flows the drilling mud 104 through the flow conduit assembly 106 at another particular (for example, pre-determined or programmed) flow rate for a particular time period to at least achieve a steady state flow at the another particular rate while measurements in steps 206 and 208 occur. This process can be repeated for any number of particular flow rates.

Method 200 can continue at step 204, which includes circulating the flow of fluid at each of the plurality of unique flow rates through one or more tubing coils of a coiled tubing assembly fluidly coupled within the flow conduit assembly between the inlet and the outlet. For example, as the flow of the drilling mud 104 circulates through the flow conduit assembly 106, the drilling mud 104 circulates through the coils 116 of the coiled tubing assembly 114. As the drilling fluid 104 circulates through the coiled tubing assembly 114, a fluid pressure of the drilling fluid 104 drops between the fluid input 120 and the fluid output 118. As in step 202, the drilling fluid 104 can be circulated at multiple, unique steady-state flow rates through the coiled tubing assembly 114.

Method 200 can continue at step 206, which includes, at each of the plurality of unique flow rates through the one or more tubing coils of the coiled tubing assembly, measuring a fluid pressure drop across the coiled tubing assembly with a pressure sensor coupled to the flow conduit assembly. For example, the pressure sensor 122 (for example, a differential pressure sensor) measures the pressure drop of the drilling fluid 104 across the coiled tubing assembly 114 at each particular, unique flow rate.

In example implementations of the rheometer system 100 in which there are multiple coiled tubing assemblies 114 (each of which including one or more coils 116), step 208 can include taking pressure drop measurements of the drilling fluid 104 across each of the multiple coiled tubing assemblies 114. In some aspects, having multiple coiled tubing assemblies 114 can allow for multiple pressure drop measurements at a particular flow rate, thereby speeding the process of obtaining many measurement pairs of flow rate and pressure drop. In some aspects, when each of the multiple coiled tubing assemblies 114 include coils 116 of differing inner diameters, measurement pairs of flow rate and pressure drop for each particular inner diameter can be obtained, thereby allowing for even more measurements for use in step 210.

Method 200 can continue at step 208, which includes, at each of the plurality of unique flow rates through the one or more tubing coils of the coiled tubing assembly, measuring a fluid pressure drop across the coiled tubing assembly with a pressure sensor coupled to the flow conduit assembly. For example, the flow meter 112 (for example, a Coriolis meter) measures the flow rate of the drilling fluid 104 to ensure or confirm that the drilling fluid 104 is at the particular, unique flow rate. As with step 206, step 208 can be repeated for each of multiple, unique flow rates of the drilling fluid 104. In some aspects, step 208 can be performed prior to or along with step 206 in method 200.

Method 200 can continue at step 210, which includes, for each unique flow rate and corresponding measured fluid pressure drop, determining one or more flow rate-dependent properties of the fluid. For example, at the completion of steps 202-208, the control system 999, for instance, has recorded or obtained multiple measurement pairs of flow rate and pressure drop (for example, with the same inner diameter of the coils 116 or with coils 116 of differing inner diameters). In some aspects, the flow rate-dependent properties of the drilling fluid 104 can be one or both of shear rate or shear stress. For example, once shear stress and shear rate are obtained for the drilling fluid 104, these properties can be plotted in order to derive other parameters of interest. Such properties can include, for example, stress and effective viscosity per strain rate, Hershel Buckley model parameters, and other rheological parameters which describe features of the shear stress versus strain rate plot for the drilling fluid 104.

In some aspects, step 210 includes determining the shear stress based on the inner diameter of the coils 116 and the measured fluid pressure drop across the coiled tubing assembly 114. For example, shear stress can be calculated by:

$${τ}_{w} = \frac{D}{4} \frac{d p}{d l}$$

where τ_{w} is the shear stress, D is the inner diameter of the one or more tubing coils 116, and

$$\frac{d p}{d l}$$

is the measured fluid pressure drop per length of the coiled tubing assembly 114. Thus, step 210 can include determining multiple shear stresses, with each determined shear stress corresponding to a particular flow rate that generates a measured fluid pressure drop and for a particular inner diameter of the coils 116.

In some aspects, the above-equation for shear stress is well suited for determining shear stress in a straight pipe for laminar flow. However, for a tubing with coils (such as coiled tubing assembly 114), a portion of the pressure drop per unit length (dp/dl) is caused from the curvature of the coils 116 and must be corrected for to correctly calculate the shear stress in the drilling fluid 104. This curvature in the coiled tubing assembly 114 can be expressed as an increase to the friction factor of the tubing. Thus, for the coiled tubing assembly 114, the pressure drop is expressed as:

$$\frac{d p}{d l} = \frac{2 f ρ {v}^{2}}{D}$$

where ƒ is the friction factor, v is the velocity of the drilling fluid 104 (determined by the flow rate and inner diameter of the tubing), and ρ is the density of the drilling fluid 104.

The friction factor in the coiled tubing assembly 114 can be corrected using the Dean number for the flow:

$$D e = R e \sqrt{\left(\frac{D}{2 R}\right)}$$

where De is the Dean number, Re is the Reynolds number, and R is the radius of the coils 116 in the coiled tubing assembly 114. In some aspects, the Reynolds number can be approximated based on expected fluid viscosity of the drilling fluid 104.

If the flow of the drilling fluid 104 is considered laminar, and the tubing of the flow conduit assembly 106 is considered smooth, the friction factor can then be calculated dependent on the value of the Dean number. For example, for De<11.6:

$$f = \frac{64}{R e}$$

For 11.6<De<2000:

$$f = \frac{\frac{64}{R e}}{1 - {\left(1 - {\left(\frac{11.6}{D e}\right)}^{.45}\right)}^{2.2}}$$

For 2000<De:

$$f = \frac{7.0144}{R e} \sqrt{D e}$$

In some aspects, Eq. 2 is not used to determine the pressure drop per unit length of the coiled tubing assembly 114 but instead, another pressure drop equation can be used that incorporates the pressure drop due to curvature, which is:

$$\frac{d p}{d l} = \frac{\frac{1}{2} {f}_{s} ρ {μ}^{2} {R}_{b} \text{Θ}}{D} + \frac{1}{2} {k}_{b} ρ {μ}^{2}$$

where R_{b} is the radius of the bend (of a coil 116), Θ is the bend angle, ƒₛ is the friction factor for a straight pipe (of the same diameter as the coil 116), µ is the dynamic viscosity of the drilling fluid 104, and k_{b} is the bend loss coefficient.

Once the shear stress is determined in step 210, the shear rate can also be determined. For example, shear rate can be determined according to the determined shear stress, velocity of the drilling fluid 104, and the inner diameter of the coiled tubing assembly 114. In some aspects, shear rate can be calculated according to:

$${γ}_{w} = \frac{1}{4} \left[3 + \frac{d \left(l n \frac{8 v}{D}\right)}{d \left({τ}_{w}\right)}\right] \left(\frac{8 v}{D}\right)$$

where γ_{w} is the shear rate and v is the velocity of the drilling fluid 104 (as determined by the flow rate and inner diameter, D).

Method 200 can continue at step 212, which includes generating a rheogram from the determined one or more flow rate-dependent properties. For example, at the conclusion of step 210, the control system 999, for example, has determined multiple values for each of the shear stress and shear rate of the drilling fluid 104. Each value of these flow rate-dependent properties can be independently calculated according to: the particular flow rate, the measured fluid pressure drop across the coiled tubing assembly 114, and the physical properties of the coiled tubing assembly 114 (for example, inner diameter etc.). Once these values have been calculated, a rheogram can be plotted, for example, graphically. In some aspects, the rheogram can include plots of shear stress vs. shear rate.

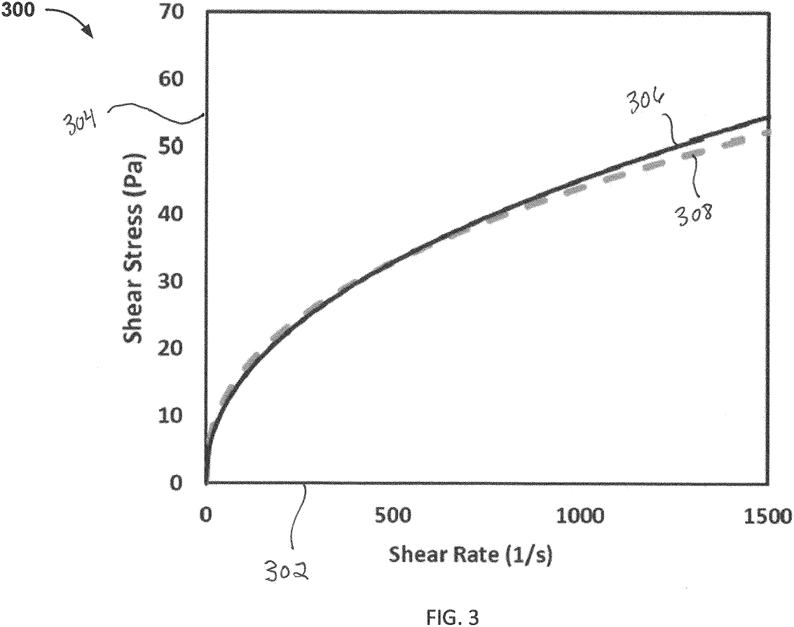

For example, FIG. 3 shows an example rheogram 300 as determined by method 200 (and the example implementation of the rheometer system 100). Rheogram 300 includes an x-axis 302 of shear rate (in s⁻¹) and a y-axis 304 of shear stress (in Pa). Curve 308 represents the charted pairs of determined shear stress v. shear rate as determined according to method 200 (and the example implementation of the rheometer system 100). As a comparison, curve 306 shows a similar sets of paired shear stress v. shear rate values as determined by a rotational viscometer.

In some aspects, method 200 can include further steps that are taken subsequently to the determination of the one or more flow rate-dependent properties and generation of the rheogram. For example, as described, further fluid properties of the drilling fluid 104 can be determined from the one or more flow rate-dependent properties and the rheogram, such as stress and effective viscosity per strain rate and Hershel Buckley model parameters. In determining the fluid properties, further steps can be taken, such as adjusting a composition of the drilling fluid 104 in order to change the measured properties. As another example, the drilling process can be adjusted based on the determined fluid properties.

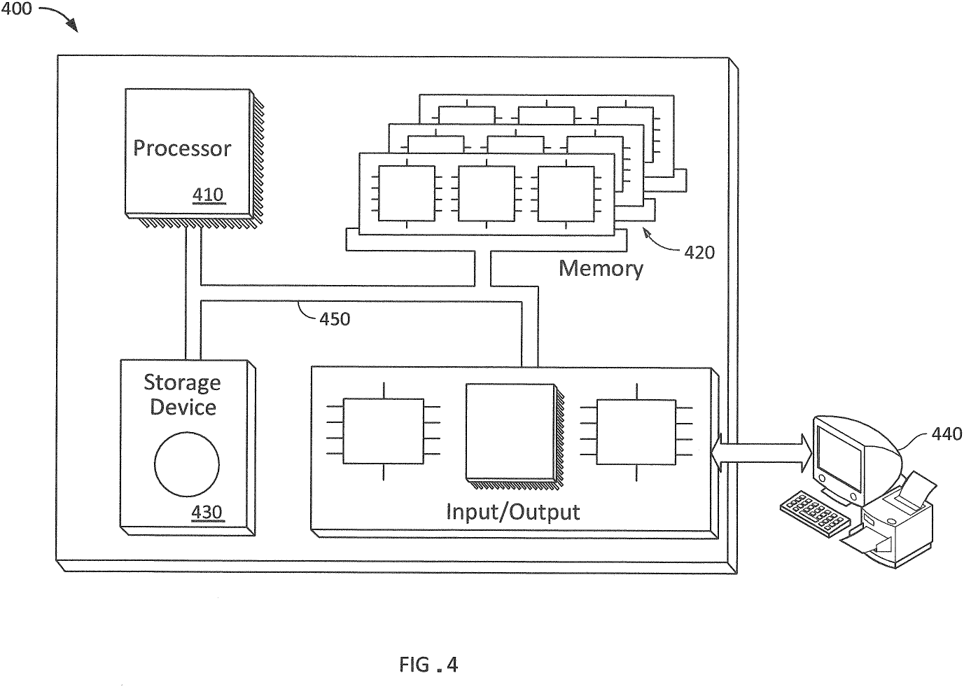

FIG. 4 is a schematic illustration of an example controller 400 (or control system) for an rheometer system, such as the rheometer system 100. For example, all or parts of the controller 400 can be used for the operations described previously, for example as or as part of the control system 999, or both. The controller 400 is intended to include various forms of digital computers, such as printed circuit boards (PCB), processors, digital circuitry, or otherwise. Additionally, the system can include portable storage media, such as, Universal Serial Bus (USB) flash drives. For example, the USB flash drives may store operating systems and other applications. The USB flash drives can include input/output components, such as a wireless transmitter or USB connector that may be inserted into a USB port of another computing device.

The controller 400 includes a processor 410, a memory 420, a storage device 430, and an input/output device 440. Each of the components 410, 420, 430, and 440 are interconnected using a system bus 450. The processor 410 is capable of processing instructions for execution within the controller 400. The processor may be designed using any of a number of architectures. For example, the processor 410 may be a CISC (Complex Instruction Set Computers) processor, a RISC (Reduced Instruction Set Computer) processor, or a MISC (Minimal Instruction Set Computer) processor.

In one implementation, the processor 410 is a single-threaded processor. In another implementation, the processor 410 is a multi-threaded processor. The processor 410 is capable of processing instructions stored in the memory 420 or on the storage device 430 to display graphical information for a user interface on the input/output device 440.

The memory 420 stores information within the controller 400. In one implementation, the memory 420 is a computer-readable medium. In one implementation, the memory 420 is a volatile memory unit. In another implementation, the memory 420 is a non-volatile memory unit.

The storage device 430 is capable of providing mass storage for the controller 400. In one implementation, the storage device 430 is a computer-readable medium. In various different implementations, the storage device 430 may be a floppy disk device, a hard disk device, an optical disk device, a tape device, flash memory, a solid state device (SSD), or a combination thereof.

The input/output device 440 provides input/output operations for the controller 400. In one implementation, the input/output device 440 includes a keyboard and/or pointing device. In another implementation, the input/output device 440 includes a display unit for displaying graphical user interfaces.

The features described can be implemented in digital electronic circuitry, or in computer hardware, firmware, software, or in combinations of them. The apparatus can be implemented in a computer program product tangibly embodied in an information carrier, for example, in a machine-readable storage device for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, solid state drives (SSDs), and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) or LED (light-emitting diode) monitor for displaying information to the user and a keyboard and a pointing device such as a mouse or a trackball by which the user can provide input to the computer. Additionally, such activities can be implemented via touchscreen flat-panel displays and other appropriate mechanisms.

The features can be implemented in a control system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include a local area network (“LAN”), a wide area network (“WAN”), peer-to-peer networks (having ad-hoc or static members), grid computing infrastructures, and the Internet.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

A number of implementations have been described. Nevertheless, it will be understood that various modifications may be made without departing from the spirit and scope of the disclosure. For example, example operations, methods, or processes described herein may include more steps or fewer steps than those described. Further, the steps in such example operations, methods, or processes may be performed in different successions than that described or illustrated in the figures. Accordingly, other implementations are within the scope of the following claims.

What is claimed is:

1. A system for determining one or more properties of a fluid, the system comprising:
a pump configured to circulate a flow of a fluid from a fluid source through a flow conduit assembly at a plurality of unique flow rates, the flow conduit assembly comprising an inlet configured to fluidly coupled to the fluid source at a first location and an outlet configured to fluidly coupled to the fluid source at a second location;
a flow meter fluidly coupled within the flow conduit assembly;
a coiled tubing assembly fluidly coupled within the flow conduit assembly between the inlet and the outlet, the coiled tubing assembly comprising one or more tubing coils;
a pressure sensor coupled to the flow conduit assembly and configured to measure a fluid pressure drop across the coiled tubing assembly; and
a control system communicably coupled to the flow meter and the pressure sensor and configured to perform operations comprising:
identifying each of the plurality of unique flow rates of the flow of fluid through the coiled tubing assembly measured by the flow meter;
identifying, for each unique flow rate, the fluid pressure drop across the coiled tubing assembly measured by the pressure sensor;
for each unique flow rate and corresponding measured fluid pressure drop, determining one or more flow rate-dependent properties of the fluid; and
generating a rheogram from the determined one or more flow rate-dependent properties.

2. The system of claim 1, wherein the fluid comprises a drilling fluid.

3. The system of claim 2, wherein the first location is at or near a bottom of a drilling fluid tank, and the second location is at or near a top of the drilling fluid tank.

4. The system of claim 1, wherein each of the plurality of unique flow rates comprises a plurality of unique steady state flow rate.

5. The system of claim 1, wherein the flow meter comprises a Coriolis meter and the pressure sensor comprises a differential pressure sensor.

6. The system of claim 1, wherein the operation of determining one or more flow rate-dependent properties of the fluid comprises:
determining, for each unique flow rate and corresponding measured fluid pressure drop, a shear rate of the fluid; and
determining, for each unique flow rate and corresponding measured fluid pressured drop, a shear stress of the fluid.

7. The system of claim 6, wherein the operation of determining the shear stress of the fluid comprises calculating the shear stress based at least in part on an inner diameter of the one or more tubing coils and the measured fluid pressure drop.

8. The system of claim 6, wherein the operation of calculating the shear stress comprises solving:
$${τ}_{w} = \frac{D}{4} \frac{d p}{d l} ,$$
where τ_{w} is the shear stress, D is the inner diameter of the one or more tubing coils, and
$$\frac{d p}{d l}$$
is the measured fluid pressure drop per length of the coiled tubing assembly.

9. The system of claim 7, wherein the operation of determining the shear rate of the fluid comprises calculating the shear rate based at least in part on the calculated shear stress, the inner diameter of the one or more tubing coils, a velocity of the flow of the fluid, and the measured fluid pressure drop.

10. The system of claim 9, wherein the operation of calculating the shear rate comprises solving:
$${γ}_{w} = \frac{1}{4} \left[3 + \frac{d \left(l n \frac{8 v}{D}\right)}{d \left({τ}_{w}\right)}\right] \left(\frac{8 v}{D}\right) ,$$
where γ_{w} is the shear rate, τ_{w} is the shear stress, D is the inner diameter of the one or more tubing coils, and v is the velocity of the fluid.

11. The system of claim 1, wherein the coiled tubing assembly comprises a first coiled tubing assembly and the one or more tubing coils comprises one or more first tubing coils, the system further comprising a second coiled tubing assembly fluidly coupled within the flow conduit assembly between the inlet and the outlet, the second coiled tubing assembly comprising one or more second tubing coils.

12. The system of claim 11, wherein the first and second coiled tubing assemblies are fluidly coupled in series.

13. The system of claim 11, wherein an inner diameter of the one or more second tubing coils is different than an inner diameter of the one or more first tubing coils.

14. The system of claim 13, wherein the control system is configured to perform operations comprising:
identifying each of the plurality of unique flow rates of the flow of fluid through the first and second coiled tubing assemblies as measured by the flow meter;
identifying, for each unique flow rate, the measured fluid pressure drop across each of the first and second coiled tubing assemblies by the pressure sensor;
for each unique flow rate and corresponding measured fluid pressure drop across the first coiled tubing assembly, determining a first set of one or more flow rate-dependent properties of the fluid;
for each unique flow rate and corresponding measured fluid pressure drop across the second coiled tubing assembly, determining a second set of one or more flow rate-dependent properties of the fluid; and
generating a rheogram from the determined first and second sets of one or more flow rate-dependent properties.

15. A method for determining one or more properties of a fluid, comprising:
circulating, with a pump, a flow of a fluid from a fluid source through a flow conduit assembly at a plurality of unique flow rates, the flow conduit assembly comprising an inlet configured to fluidly coupled to the fluid source at a first location and an outlet configured to fluidly coupled to the fluid source at a second location;
circulating, with the pump, the flow of fluid at each of the plurality of unique flow rates through one or more tubing coils of a coiled tubing assembly fluidly coupled within the flow conduit assembly between the inlet and the outlet;
during circulation of the flow of fluid at each of the plurality of unique flow rates through the one or more tubing coils of the coiled tubing assembly, measuring the unique flow rate with a flow meter coupled within the flow conduit assembly;
during circulation of the flow of fluid at each of the plurality of unique flow rates through the one or more tubing coils of the coiled tubing assembly, measuring a fluid pressure drop across the coiled tubing assembly with a pressure sensor coupled to the flow conduit assembly;
for each unique flow rate and corresponding measured fluid pressure drop, determining one or more flow rate-dependent properties of the fluid; and
generating a rheogram from the determined one or more flow rate-dependent properties.

16. The method of claim 15, wherein the fluid comprises a drilling fluid.

17. The method of claim 16, wherein the first location is at or near a bottom of a drilling fluid tank, and the second location is at or near a top of the drilling fluid tank.

18. The method of claim 15, wherein each of the plurality of unique flow rates comprises a plurality of unique steady state flow rate.

19. The method of claim 15, wherein the flow meter comprises a Coriolis meter and the pressure sensor comprises a differential pressure sensor.

20. The method of claim 15, wherein determining one or more flow rate-dependent properties of the fluid comprises:
determining, for each unique flow rate and corresponding measured fluid pressure drop, a shear rate of the fluid; and
determining, for each unique flow rate and corresponding measured fluid pressured drop, a shear stress of the fluid.

21. The method of claim 20, wherein determining the shear stress of the fluid comprises calculating the shear stress based at least in part on an inner diameter of the one or more tubing coils and the measured fluid pressure drop.

22. The method of claim 20, wherein calculating the shear stress comprises solving:
$${τ}_{w} = \frac{D}{4} \frac{d p}{d l} ,$$
where τ_{w} is the shear stress, D is the inner diameter of the one or more tubing coils, and
$$\frac{d p}{d l}$$
is the measured fluid pressure drop per length of the coiled tubing assembly.

23. The method of claim 22, wherein determining the shear rate of the fluid comprises calculating the shear rate based at least in part on the calculated shear stress, the inner diameter of the one or more tubing coils, a velocity of the flow of the fluid, and the measured fluid pressure drop.

24. The method of claim 23, wherein calculating the shear rate comprises solving:
$${γ}_{w} = \frac{1}{4} \left[3 + \frac{d \left(l n \frac{8 v}{D}\right)}{d \left({τ}_{w}\right)}\right] \left(\frac{8 v}{D}\right) ,$$
where γ_{w} is the shear rate, τ_{w} is the shear stress, D is the inner diameter of the one or more tubing coils, and v is the velocity of the fluid.

25. The method of claim 15, wherein the coiled tubing assembly comprises a first coiled tubing assembly and the one or more tubing coils comprises one or more first tubing coils, the method further comprising:
circulating, with the pump, the flow of fluid at each of the plurality of unique flow rates through one or more second tubing coils of a second coiled tubing assembly fluidly coupled within the flow conduit assembly between the inlet and the outlet.

26. The method of claim 25, wherein the first and second coiled tubing assemblies are fluidly coupled in series.

27. The method of claim 25, wherein an inner diameter of the one or more second tubing coils is different than an inner diameter of the one or more first tubing coils.

28. The method of claim 27, further comprising:
during circulation of the flow of fluid at each of the plurality of unique flow rates through the one or more first tubing coils of the first coiled tubing assembly and the one or more second tubing coils of the second coiled tubing assembly, measuring the unique flow rate with the flow meter coupled within the flow conduit assembly;
during circulation of the flow of fluid at each of the plurality of unique flow rates through the one or more first tubing coils of the first coiled tubing assembly, measuring a first set of fluid pressure drops across the first coiled tubing assembly with the pressure sensor coupled to the flow conduit assembly;
during circulation of the flow of fluid at each of the plurality of unique flow rates through the one or more second tubing coils of the second coiled tubing assembly, measuring a second set of fluid pressures drop across the second coiled tubing assembly with the pressure sensor coupled to the flow conduit assembly;
for each unique flow rate and corresponding measured first and second sets of fluid pressure drops, determining one or more flow rate-dependent properties of the fluid; and generating a rheogram from the determined one or more flow rate-dependent properties.
